(19) United States
(12) Patent Application Publication
(10) Pub. No.: US 2020/0281666 A1
(43) Pub. Date: Sep. 10, 2020

(54) STEERABLE CATHETER FLEXIBLE ROBOTIC SYSTEM FOR USE WITH ENDOSCOPES

(71) Applicant: The Regents of the University of California, Oakland, CA (US)

(72) Inventors: Aaron Gunn, La Jolla, CA (US); Michael Yip, La Jolla, CA (US); Philip WEISSBROD, La Jolla, CA (US)

(21) Appl. No.: 16/652,968

(22) PCT Filed: Oct. 2, 2018
(86) PCT No.: PCT/US2018/053952

§ 371 Date: Apr. 1, 2020

Related U.S. Application Data
(60) Provisional application No. 62/567,057, filed on Oct. 2, 2017.

(51) Int. Cl. A61B 34/30; A61B 34/35; A61B 34/00; A61B 17/00; A61B 1/05; A61B 1/018

(52) U.S. Cl. CPC A61B 34/30; A61B 34/35; A61B 34/71; A61B 17/00234; A61M 25/0026; A61B 1/018; A61B 2034/303; A61B 2034/301; A61B 2017/0034; A61B 1/05

(57) ABSTRACT

A surgical arrangement includes an endoscope having an insertion tube with an imaging system disposed on its distal end and at least one instrument channel extending therethrough. A catheter subsystem of a steerable catheter robotic system is removably insertable into the instrument channel. The catheter subsystem includes a flexible outer sheath having a proximal end and a distal end. At least one flexible multi-lumen assembly extends through the outer sheath. The multi-lumen assembly has a proximal end and a distal end. A robotic instrument for performing a surgical procedure is operatively and removably attachable to the distal end of the multi-lumen assembly such that the robotic instrument is teleoperable

### CROSS-REFERENCE TO RELATED APPLICATIONS

This application claims benefit of priority of U.S. Provisional Patent Application Ser. No. 62/567,057, filed Oct. 2, 2017, entitled “SINGLE CATHETER FLEXIBLE ROBOTIC SYSTEM”, owned by the assignee of the present application and herein incorporated by reference in its entirety.

### BACKGROUND

Remotely-controlled surgical instruments, which can include teleoperated surgical instruments (e.g., surgical instruments operated at least in part with computer assistance, such as instruments operated with robotic technology) as well as manually operated (e.g., laparoscopic, thorascopic) surgical instruments, are often used in minimally invasive medical procedures. During such procedures, a surgical instrument, which may extend through a cannula inserted into a patient's body, can be remotely manipulated to perform a procedure at a surgical site. For example, in a teleoperated surgical system, cannulas and surgical instruments can be mounted at manipulator arms of a patient side cart and be remotely manipulated via teleoperation at a surgeon console.

In the present landscape of surgical robotics, the field of continuum (or flexible) surgical robotic systems is still very much in development. These biomimetic systems, many modeled after tentacles or trunks, allow for minimally-invasive access to previously unreachable anatomy. By developing these devices with a smaller footprint and more robust laminate manufacturing techniques, lower impact surgery can be portably and efficiently performed in even tighter spaces than traditional rigid surgical robots.

### SUMMARY

In accordance with one aspect of the present disclosure, a minimally invasive surgical arrangement is presented with enables dexterious instrument control in tight spaces and distal anatomy, which can give rise to reduced procedure times, recovery periods and morbidity. A steerable catheter robotic system is used in conjunction with any suitable type of endoscope to provide additional degrees of freedom in the surgical field, giving operators the ability to navigate around anatomy more freely, as well as providing access to difficult to reach anatomic regions, thus achieving all the benefits of minimally invasive surgery in a wide range of scenarios, without incurring significant medical cost or requiring specialized facilities and personnel resources.

In one particular embodiment, the surgical arrangement includes an endoscope having an insertion tube with an imaging system disposed on its distal end and at least one instrument channel extending therethrough. A catheter subsystem of a steerable catheter robotic system is removably insertable into the instrument channel. The catheter subsystem includes a flexible outer sheath having a proximal end and a distal end. At least one flexible multi-lumen assembly extends through the outer sheath. The multi-lumen assembly has a proximal end and a distal end. A robotic instrument for performing a surgical procedure is operatively and removably attachable to the distal end of the multi-lumen assembly such that the robotic instrument is teleoperable

### BRIEF DESCRIPTION OF THE DRAWINGS

FIGS. 1 and 2 show perspective views of a multi-catheter subsystem.

FIG. 3 shows one example of a multi-lumen assembly that is used to steer a single one of the robotic instruments shown in FIGS. 1 and 2.

FIG. 4 shows a motor control assembly.

FIG. 5 shows a pully housing assembly.

FIG. 6 shows an example of a steerable catheter robotic system that includes the multi-catheter subsystem shown in FIGS. 1 and 2.

FIGS. 7 and 8 illustrate a perspective view and a perspective cutaway view, respectively, of one example of an endoscope with which the steerable catheter robotic system shown herein may be employed.

FIG. 9 shows one example of a surgical arrangement that includes an endoscope and the steerable catheter robotic system.

### DETAILED DESCRIPTION

As described in more detail below, a steerable catheter robotic system with a significantly reduced size-footprint is provided for deployment in field or outpatient pulmonary surgical procedures. The small size and portability of this system can help overcome a major disadvantage of current surgical robots which take up an immense amount of space in already crowded-operating rooms, while still being able to imitate, copy and improve human capabilities. In some embodiments the dimensions of the robotic instruments or tools may be as small as 1 mm.

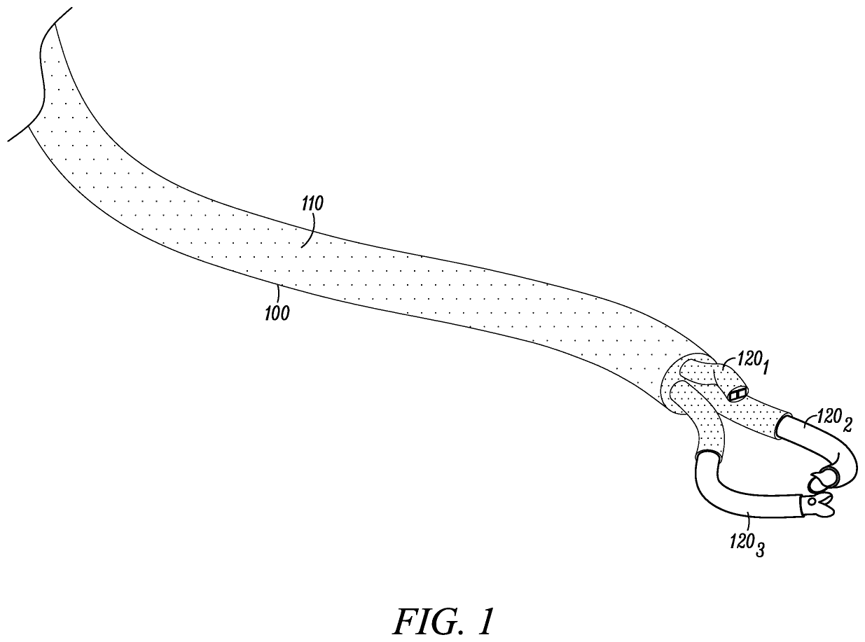

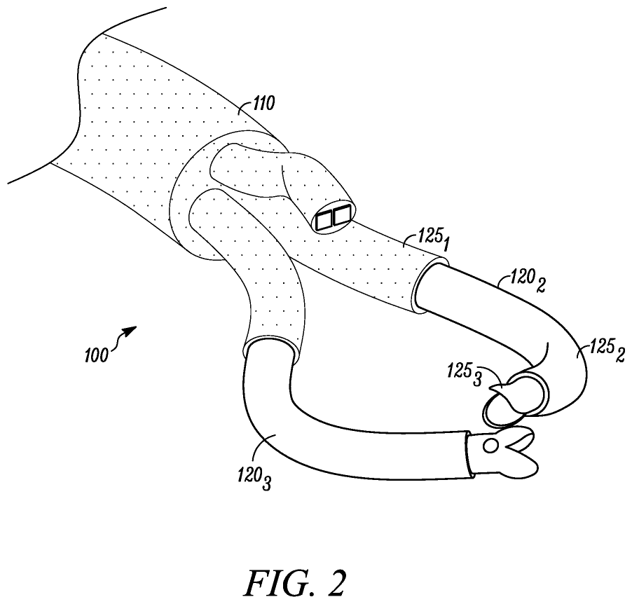

FIGS. 1 and 2 show perspective views of a multi-catheter subsystem 100 that includes a flexible outer guide shaft 110 having a distal end from which one or more robotic instruments extend. Although the embodiment shown in FIGS. 1 and 2 shows three robotic instruments 120₁, 120₂ and 120₃ (“120”), more generally any number of such robotic instruments 120 may be employed. In this particular embodiment the robotic instruments 120 include a camera 120₁ and first and second grasping forceps 120₂ and 120₃. A control assembly (not shown in FIGS. 1 and 2) is located at the proximal end of the outer sheath 110 controls the operation of the robotic instruments 120.

In some embodiments each robotic instrument 120 may include two or more articulating segments that provide the instrument with multiple degrees of freedom. For instance, as best seen in FIG. 2, the first grasping forcep 120₂ includes three articulating segments 125₁, 125₂ and 125₃. The second grasping forcep 120₃ may be similarly configured. By employing a suitable number of articulating segments, some instruments may be supplied with 7 degrees of freedom of articulation (i.e. positional control of x, y, z in cartesian space, and roll-pitch-yaw in orientation, and an actuation degree of freedom such as a pinch grip of a forcep), thereby essentially recovering the dexterity of a human hand. In such an embodiment a one-to-one mappings can be advantageously realized of a teleoperating using to the robotic instrument. If more than 7 degrees of freedom are provided to a given instrument, the instrument can have additional degrees of freedom to conform to the environment without affecting the controllability of the 7 degrees of freedom that are controlled by the human operator. Some instruments may have additional elbow deflection locations that allow the shape of the instrument to better conform to the environment.

When one of the robotic instruments is a camera, it may be operated with only 6 degrees of freedom for full visual control, although the focal depth (if so integrated) may be considered a 7^{th} degree of freedom.

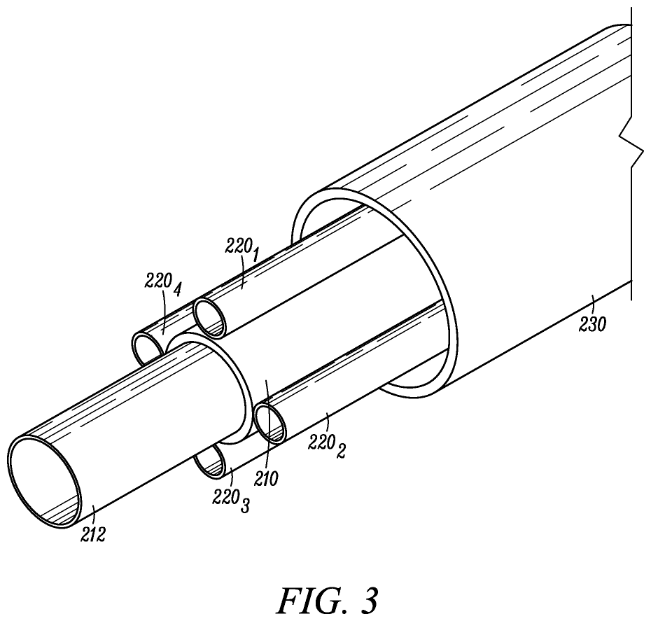

FIG. 3 shows one example of a multi-lumen assembly 200 that is used to steer a single instrument 120. Each of the lumens is formed from a flexible material such as a flexible polymer. The multi-lumen assembly 200 extends through the outer guide shaft 110 shown in FIG. 1. The multi-lumen assembly 200 includes a center channel 210 that has a liner 212 that serves as an instrument port for one segment of a multi-segment instrument (or a complete instrument of a single-segment instrument). Surrounding the center channel 210 are a series of control lumens 220 through which articulation wires (not shown) extend. In this example 4 control lumens 220₁, 220₂, 220₃ and 220₄ are shown. The control lumens 220 are secured (e.g., fused) to the center channel 210. Articulation of the instrument segment located in the center channel 210 is determined by the coordinated operation of the articulation wires via the control assembly, which will be described below. The center channel 210 and the control lumens 220 may extend through a flexible sheath 230 (which itself extends through the outer guide shaft 110 shown in FIGS. 1 and 2).

Each articulating segment of a multi-segment instrument includes its own dedicated multi-lumen assembly 200 for controlling that segment. The different multi-lumen assemblies 200 of a single multi-segment instrument may be concentrically arranged with one another.

As mentioned above, the multi-lumen assembly 200 may be fabricated from flexible polymers. For example, in some embodiments the flexible sheath 230 and center channel 210 may be formed from a varying durometer thermoplastic polymer such as a polyester block amide (available, for instance, under the tradename PEBAX®). An optional stainless steel or fiber braid (not shown) may surround the flexible sheath 230. Likewise, in some embodiments the control lumens may be formed polymide and the liner 212 lining the center channel 210 may be formed from PTFE (i.e., Teflon®). The use of flexible polymers for the multi-lumen assembly affords significant flexibility in short segments without deterioration of the assembly and tight radiuses of curvatures can be achieved. Lamination of these polymers, which can become micron-thickness layers, enables these robotically controlled lumens to reach as small as 1 mm in diameter.

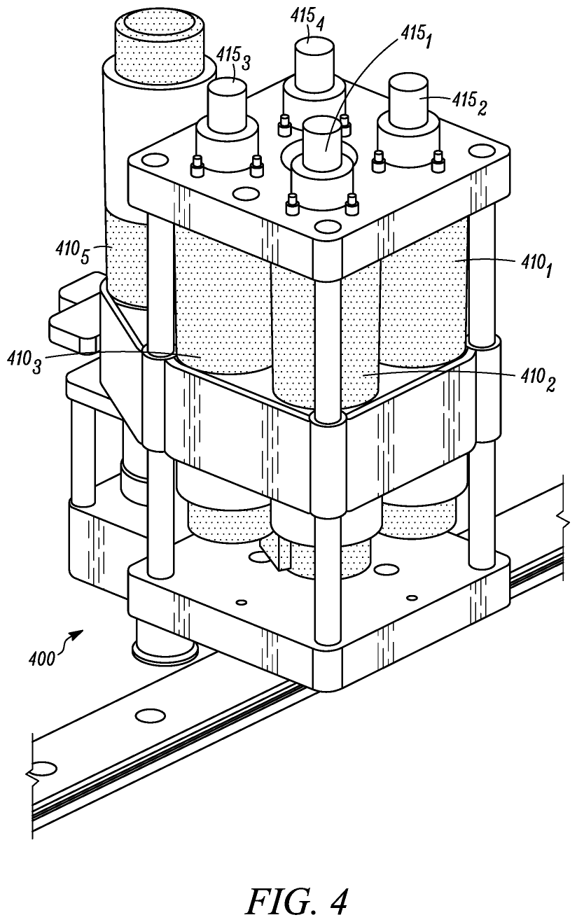

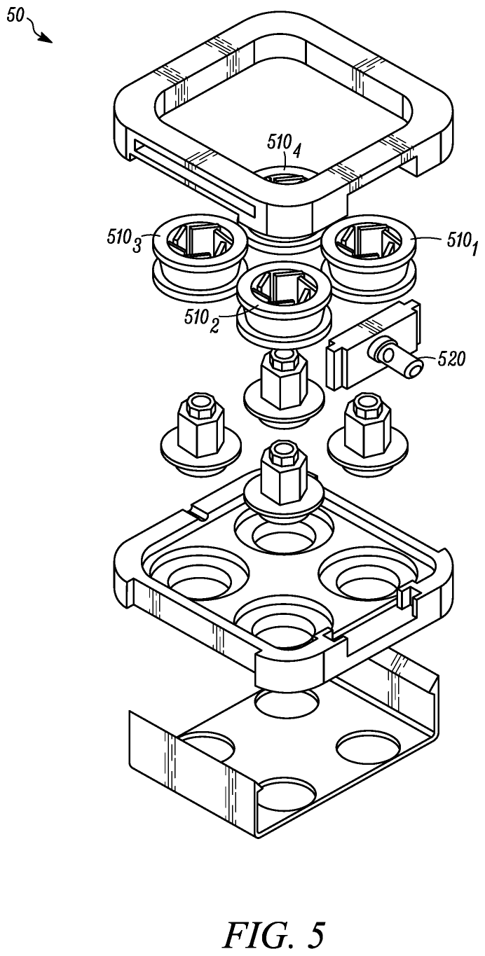

FIG. 4 shows a motor control assembly 400 that can be used in conjunction with a pully housing assembly 500 (FIG. 5) to control the four pull wires that extend through the control lumens 220. The motor control assembly 400 includes four motors 410₁, 410₂, 410₃ and 410₄ (where motor 410₄ is not visible in FIG. 4). that each respectively control the rotation of a rotatable shaft 415₁, 415₂, 415₃ and 415₄. The pully housing assembly 500 includes four torque-limiting pulleys 510₁, 510₂, 510₃ and 510₄. When the pulley housing assembly 500 is mated with the motor control assembly 400 each pulley 510₁, 510₂, 510₃ and 510₄ is axially mounted on one of the shafts 415₁, 415₂, 415₃ and 415₄. The pulley housing assembly 500 also includes a shaft mount 520 onto which is mounted the outer guide shaft 110 and the multi-lumen assemblies 200 extending therethrough. Once installed, rotational actuation of the motors 410 located in the motor control assembly 420 is translated to linear actuation, providing four degrees of freedom to each instrument segment.

The motor control assembly 400 includes an additional motor 4105 that is used to extend and retract the robotic instrument under its control.

The control of the robotic instruments is accomplished using inverse kinematics to map Cartesian coordinates into the positions of the four pull wires. Coordinates are first multiplied by a dynamically adjustable rotation matrix, and then by constants derived during a simple calibration process in order to standardize actuation across multiple instruments. A position-based control approach using analog values to scale targets in Cartesian space that are then mapped to R⁴, resulting in high position accuracy along with precise control over actuation velocity. The final result is accurate and intuitive control over two degrees of freedom per instrument, all mapped to a user interface.

More specifically, the exact mapping between a deflection and the amount of displacement of the articulation wires is a nonlinear mapping,

x(s)=f(q₁,q₂ . . . qₘ)

where x represents a distal deflection specified as the curvature from proximal (0) to distal (s) end, (s: 0→total_length), and q₁ . . . qₘ represents the displacements of m wires. The nonlinear mapping f may be known a-priori based on geometric or mechanic reasoning, or the mapping may be found using a regression strategy (such as a least-squares fit, or neural network approach). With the mapping, a desired shape x(s) may be found by taking the inverse mapping f⁻¹ which can be found either analytically, if possible, or empirically using gradient descent.

q₁, . . . qₘ=f⁻¹(x(s))

This mapping and inverse mapping may be performed by any suitable processor.

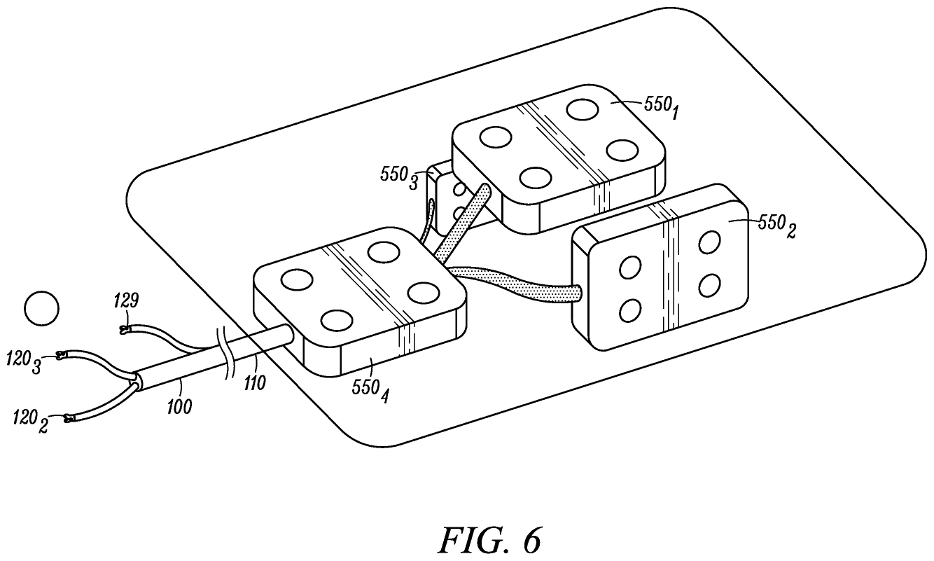

FIG. 6 shows an example of the steerable catheter robotic system that includes the multi-catheter subsystem 110 shown in FIGS. 1 and 2, which includes the three instruments 120₁, 120₂ and 120₃. Like reference numerals shown in FIG. 6 and the remaining figures denote like elements. As shown, the proximal end of the multi-catheter subsystem 110 includes controllers 550₁, 550₂, 550₃ and 550₄ (“550”). Each controller 550 includes one of the motor control assemblies 400 mated with one of the pulley housing assemblies 500. Controller 550₁ is used to control instrument 120₁, controller 550₂ is used to control instrument 120₂ and controller 550₃ is used to control instrument 120₃. The additional controller 550₄ is used control the overall movement of the multi-catheter subsystem 110.

Control of the steerable catheter robotic system via a user interface (not shown) focuses on two distinct tasks: robot movement and multiple catheter articulation. Both movements can be controlled from a single console. For instance, in one embodiment the operator is able to advance the robot via a haptic joystick. The path of the multi-catheter subsystem can be visualized on a display of the user interface console. The display may include a high-definition or 3-D screen. Additional screens within the console may allow for projection of imaging studies or electromagnetic instrument registration for use during the procedure being performed. The joystick allows forward and backward movement and 180° movement in an x and y plane of the distal tip. To prevent traumatic navigation, haptic feedback may be provided which is associated with the platform movement. Once positioned in the desired location, the platform can be fixed to allow stability during instrument insertion and movement.

In some cases the desired path to be traversed by the catheter robotic system may be specified by the operator using a component of the user interface (e.g., a joystick, mouse, drawing pad). This information is used as input to the above-mentioned inverse mapping process and the results are delivered to the motors that drive the articulation wires in the catheter robotic system. In other circumstances, instead of specifying the path to be traversed by the catheter robotic system, it may be desirable to specify the final distal position of the catheter and allow the processor to resolve the path that should be followed to reach that final position.

As discussed above, in one embodiment there are two articulating instruments that can be inserted through the length of the multi-catheter subsystem. Movement of each instrument is controlled by independent finger grasping interfaces. The instruments can be advanced or withdrawn by depression or retraction of a grasping unit. In instances where there are no grasping movements, the instruments may be moved as if grasping a virtual pencil.

A wide variety of different interchangeable robotic instruments may be used in the multi-catheter subsystem. Examples of such instruments include, without limitation, biopsy cups, grasping forceps, injection needles, biopsy needles, laser introducers, basket retrievers, hot knives, clip appliers, and scissors. The instrument or instruments that are used will be application-dependent. Examples of such applications include laryngeal, pharyngeal, hypopharyngeal, tracheal, bronchial, esophageal, stomach, large and small bowel applications. Additionally, applications include newer advanced endoscopic procedures, including endoluminal tumor ablation in varying anatomic locations, Peroral Endoscopic Myotomy (POEM), and Natural Orifice Transluminal Endoscopic Surgery (NOTES).

Robotic instruments may be interchangeable so that the multi-catheter subsystem 100 can swap the types and locations of instruments as required to generate different configurations for a user to extend their ability to work with tissues in a narrow space, extend their reach, improve their visual range, or improve the ergonomics of control. The software controlling the multi-catheter sub-system may reposition its coordinate frame to match an intuitive viewpoint of the teleoperator.

In some cases it is possible that the system can introduce more robotic instruments than a single user can control. In this scenario, both a primary user and an assistant may operate different instruments through the same system, enabling multiple robotic instruments to be controlled simultaneously. This encourages shared tasks, allowing assistants to help with the retraction of objects or environmental roadblocks while the primary user is operating on the exposed area.

One embodiment of the system may involve the autonomous control of one instrument that follows or performs some assistive task that follows the behavior of a primary user. For example, a continuous ablation using a laser that reaches deeper within a site may be realized by having one of the robotic instruments follow a user-controlled ablation probe as it moves through the environment, i.e., a robotically controlled camera. In this case one instrument would be teleoperated while the other is autonomous and following the teleoperated camera.

The ability to simultaneously control and steer multiple robotic instruments can provide critical capabilities in manipulating areas of tissues with bimanual manipulation. For example, controlled stretching of tissue or peeling of tissue can be achieved only with two or more instruments. Likewise, the ability to mount and control a camera independently of the other instruments (and vice-versa) is a significant advantage over current endoscopic approaches where the endoscope is the camera and dictates the controllability of the instruments exiting from its orientation-fixed instrument lumen. Moreover, the multi-catheter system may be mixed with manual instrumentation given that the instrumentation fits within the available lumens for control.

Another advantage of the steerable catheter robotic system described herein is that one of its intracorporeal instruments can be used to stabilize another when there is a desire for improved stiffness. For example, an outstretched robotic instrument may become too compliant to lift a tissue that is far away. A support provided from a second robotic instrument may be devised to generate mechanical leverage that can amplify the force generation or the reachability of the original, unsupported instrument. In the same way, the robotic instruments may be used to support the sub-system in general and create anchors to provide stabilization against patient or anatomical motions or more generally to combat moment-arm effects.

Yet another advantage of the steerable catheter robotic system described herein arises in those embodiments that are fabricated exclusively from polymer or other non-metallic materials since these embodiments may be used in conjunction with magnetic resonance imaging (MRI) techniques.

In some embodiments, the steerable catheter robotic system is configured to be delivered through the working or instrument channel of a wide variety of different endoscopes. Such endoscopes can provide a way to navigate tortuous native patient cavities, but require a certain level of rigidity and size, making them less than ideal to navigate within small spaces once a surgical site is reached. Conventional tools and instruments that are designed to pass through conventional instrument channels of an endoscope are often more flexible, but generally only axial motion is controllable. By using the steerable catheter robotic system described herein, a substantial increase in instrument dexterity and reach can be achieved, while minimizing costs and equipment traditionally associated with robot-assisted procedures. The added dexterity provided to the operator while working in a very limited space results in a substantial broadening of the types of procedures that can be performed through a flexible endoscope. Moreover, the robotic steering of small caliber tools enables access to deeper anatomic structures than has previously been possible.

In order to be used in an endoscope, the steerable catheter robotic system needs to have a sufficiently small diameter so that it fits through conventional instrument channels, which typically have diameters ranging from ______ to ______. To accomplish this it will generally be necessary to limit the number of robotic instruments that may be used in the steerable catheter robotic system. For instance, in some cases the steerable catheter robotic system may be limited to only a single robotic instrument with 1 or 2 segments. Such a system will generally be able to be accommodated through the instrument channel of most typical endoscopes.

In use, the steerable catheter robotic system may be sufficiently light that the entire assembly, including the motor control assembly and the pulley housing assembly, may be handheld. In other cases the steerable catheter robotic system may be clamped or otherwise secured to an articulating support arm to support its weight.

There are many types of endoscopes, and they are generally named in relation to the organs or areas with which they are used. For example, gastroscopes are used for examination and treatment of the esophagus, stomach and duodenum; colonoscopes for the colon; bronchoscopes for the bronchi; laparoscopes for the peritoneal cavity; sigmoidoscopes for the rectum and the sigmoid colon; arthroscopes for joints; cystoscopes for the urinary bladder; and angioscopes for the examination of blood vessels. Embodiments of the steerable catheter robotic system shown herein may be used in conjunction with any of these different types of endoscopes. Moreover, the steerable catheter robotic system is not limited to medical applications but may be used in conjunction with other types of endoscopes such as borescopes.

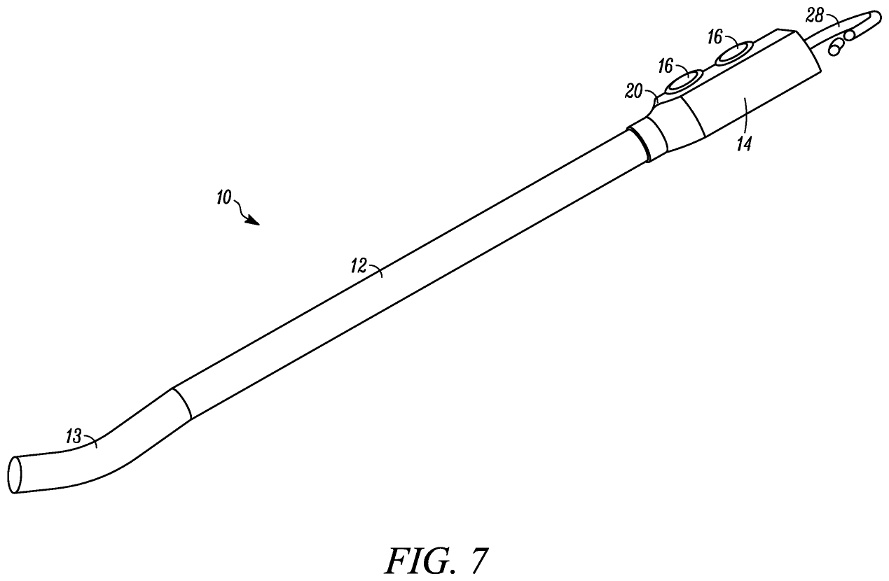

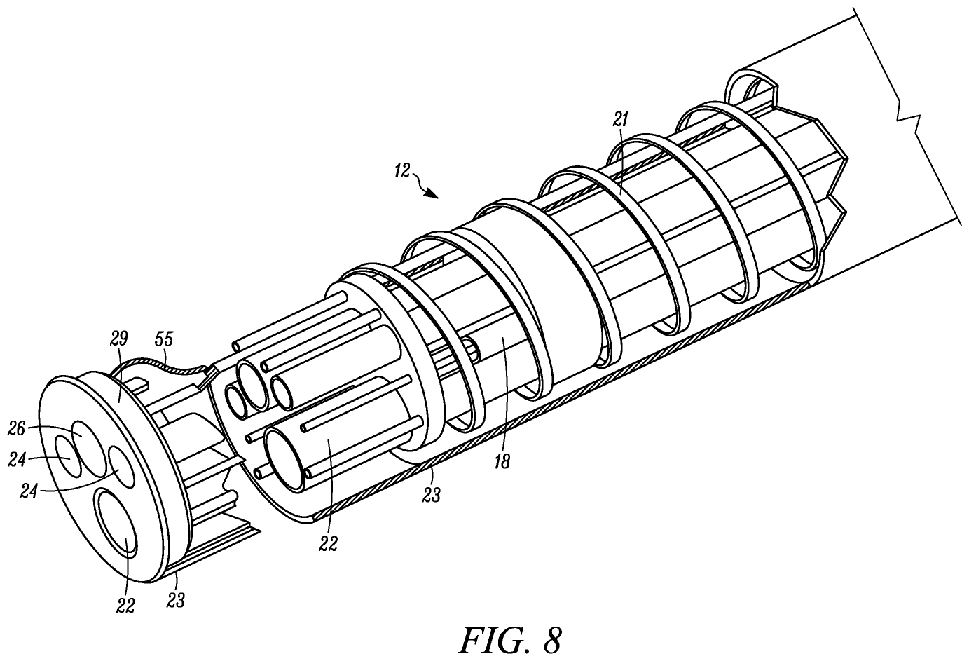

FIGS. 7 and 8 illustrate a perspective view and a perspective cutaway view, respectively, of one example of an endoscope with which the steerable catheter robotic system shown herein may be employed. The endoscope 10 can be used in a variety of medical procedures in which imaging of a body tissue, organ, cavity or lumen is required.

The endoscope 10 includes an insertion tube 12 having a imaging device 26 at its distal end (FIG. 8) and a control handle 14 connected to the insertion tube 12. The insertion tube 12 may be detachable from the control handle 14 or may be integrally formed with the control handle 14. The diameter, length and flexibility of the insertion tube 12 depend on the procedure for which the endoscope 10 is used.

As shown in FIG. 8, the insertion tube 12 has one or more longitudinal channels 22 through which an instrument can reach the body cavity to perform any desired procedures. One of the channels 22 can be used to deliver the steerable flexible catheter described herein. The insertion tube 12 may be steerable or have a steerable distal end region 13 (FIG. 7). The insertion tube 12 also may have control cables 18 (FIG. 8) for the manipulation of the insertion tube 12. The control cables 18 are symmetrically positioned within the insertion tube 12 and extend along the length of the insertion tube 12. The control cables 18 may be anchored at or near the distal end of the insertion tube 12. The control cables 18 are attached to controls (not shown) in the handle 14. Using the controls, the wires can be pulled to bend the distal end region 13 of the insertion tube 12 in a given direction.

The imaging device 26 at the distal end of the insertion tube 12 may include, for example, a lens, single chip sensor, multiple chip sensor or fiber optic implemented devices. The imaging device 26, in electrical communication with a processor and/or monitor, may provide still images or recorded or live video images. In addition to the main imaging device 26, the distal end of the insertion tube 12 may include one or more light sources 24, such as light emitting diodes (LEDs) or fiber optical delivery of light from an external light source.

As shown in FIG. 8, the insertion tube 12 may include a flexible ribbon coil 21 and a flexible sheath 23 that is used to protect the internal components of the insertion tube 12, such as the channels 22, wires and cables 18, from the environment of the body. The end cap 29 of the insertion tube 12 seals the open end of the sheath 23 to close the distal end of the insertion tube 12. The end cap 29 includes an exit port for the channel 22 and peripheral metal posts or sockets (not shown) to which the wires of the control cables 18 are attached.

As shown in FIG. 7, the control handle 14 may include one or more control knobs 16 that are attached to control cables 18 (FIG. 2) for the manipulation of the insertion tube 12. The control handle 14 has one or more ports and/or valves 20 for controlling access to the channels 22 (FIG. 8) of the insertion tube 12. One of the ports may be used for the insertion of the steerable robotic catheter described herein.

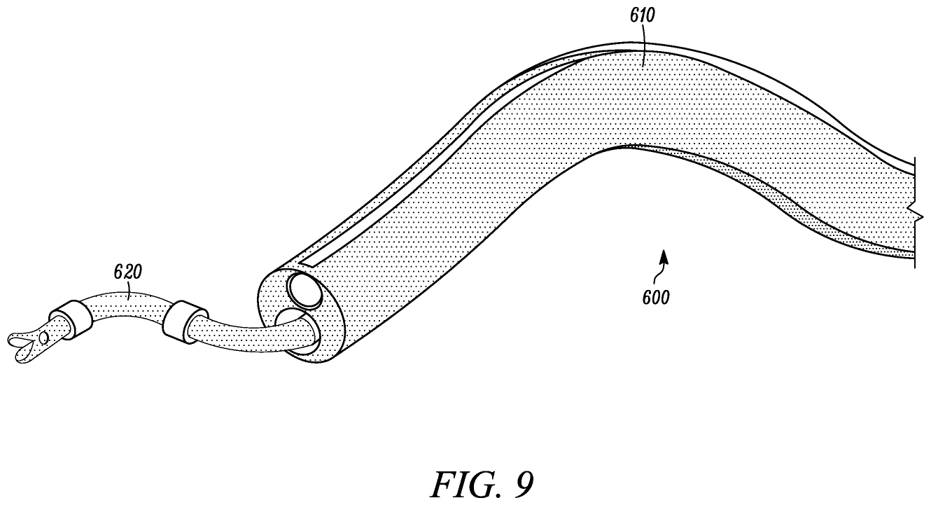

FIG. 9 shows one example of a surgical arrangement 600 that includes an endoscope and the steerable catheter robotic system. The figures show the insertion tube 610 of the endoscope through which the distal end 620 of steerable catheter robotic system is visible.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

1. A surgical arrangement, comprising:
an endoscope having an insertion tube with an imaging system disposed on its distal end and at least one instrument channel extending therethrough;
a catheter subsystem of a steerable catheter robotic system that is removably insertable into the instrument channel, the catheter subsystem comprising:
a flexible outer sheath having a proximal end and a distal end;
at least one flexible multi-lumen assembly extending through the outer sheath, the multi-lumen assembly having a proximal end and a distal end;
a robotic instrument for performing a surgical procedure, the robotic instrument being operatively and removably attachable to the distal end of the multi-lumen assembly such that the robotic instrument is teleoperable.

2. The surgical arrangement of claim 1 wherein the at least one flexible multi-lumen assembly includes a plurality of flexible multi-lumen assemblies and the robotic instrument includes a plurality of interconnected articulating segments, each of the articulating segments being operatively and removably attachable to a different one of the multi-lumen assemblies.

3. The surgical arrangement of claim 2 wherein the instrument is configured to have 7 degrees of freedom.

4. The surgical arrangement of claim 1 wherein the flexible outer sheath, the flexible multi-lumen assembly and robotic instrument are formed from polymer materials.

5. The surgical arrangement of claim 1 wherein the at least one multi-lumen assembly includes at least one actuating arrangement for steering the instrument attached thereto.

6. The surgical arrangement of claim 5 further comprising a control assembly operatively coupled to the proximal lend of the at least one multi-lumen assembly for providing rotational movement that imparts translational movement to the actuating arrangement.

7. The surgical arrangement of claim 6 wherein at least one of the actuating arrangements includes:
a plurality of control lumens attached to and surrounding a central lumen to which one of the instruments is removably attached; and
a plurality of pull wires each extending through one of the control lumens, a proximal end of each of the pull wires being operatively connected to the control assembly.
